(19) United States
(12) Patent Application Publication
(10) Pub. No.: US 2019/0336163 A1
(43) Pub. Date: Nov. 7, 2019

(54) DEVICES, SYSTEMS, AND METHODS FOR PERCUTANEOUS TRANS-SEPTAL PUNCTURE

(71) Applicant: Corvia Medical, Inc., Tewksbury, MA (US)

(72) Inventors: Edward I. McNamara, Chelmsford, MA (US); Matthew J. Finch, Reading, MA (US); Stephen J. Forcucci, Winchester, MA (US); Carol A. Devellian, Topsfield, MA (US)

(73) Assignee: Corvia Medical, Inc., Tewksbury, MA (US)

(21) Appl. No.: 16/510,925

(22) Filed: Jul. 14, 2019

Related U.S. Application Data
(63) Continuation of application No. 14/207,494, filed on Mar. 12, 2014, now abandoned.
(60) Provisional application No. 61/789,519, filed on Mar. 15, 2013.

(51) Int. Cl. A61B 17/34

(52) U.S. Cl. CPC A61B 17/3403; A61B 2090/033; A61B 17/3478

(57) ABSTRACT

The present teachings provide devices for puncturing the atrial septum percutaneously. Specifically, one aspect of the present teachings provides a trans-septal puncturing device comprising one or more of a locating element, a stabilizing element, a puncturing element, a safety element, and optionally a monitoring element, each of which is disposed in a lumen of a sheath. The locating element and the stabilizing element locate a desired puncture site and stabilize the trans-septal puncturing device at or near the site. The puncturing element of the trans-septal puncturing device has a limited puncturing distance due in certain instances to the safety element. The present teachings provide methods of using such device for percutaneously locating a fossa ovalis, stabilizing such device at the fossa ovalis, and piercing tissue across the fossa ovalis.

### FIELD OF THE INVENTION

The present teachings relate to devices and methods that allow a clinician to identify an optimal puncture site, i.e. the fossa ovalis, and create an aperture at such a site.

### BACKGROUND

The goal of a trans-septal catheterization is to cross from the right atrium to the left atrium through the fossa ovalis. In approximately 10% of the patients, this maneuver is performed during a right heart catheterization with a woven Dacron catheter because of the presence of a patent foramen ovale. But for the rest of the patients, a puncture with a needle and catheter combination is required to access the left atrium.

The fossa ovalis is posterior and caudal to the aortic root and anterior to the free wall of the right atrium. The fossa ovalis is located superiorly and posteriorly to the ostium of the coronary sinus and posterior of the tricuspid annulus and right atrial appendage. The fossa ovalis itself is approximately 2 cm in diameter and is bounded superiorly by a ridge—the limbus.

A typical trans-septal catheterization is performed from the right femoral vein, although transjugular techniques have also been used. For the femoral approach, a 70-cm curved Brockenbrough needle, which tapers from 18 gauges to 21 gauges at the tip, is used. In general, the progress of the needle tip is monitored fluoroscopically. In some instances, the progress of the needle tip is monitored by intracardiac echocardiography.

Although puncture of the fossa ovalis itself is quite safe, the danger of a trans-septal puncture lies in the possibility that the needle and catheter inadvertently puncture an adjacent structure (i.e., the posterior wall of the right atrium, the coronary sinus, or the aortic root). For example, when the septum bulges towards the right atrium, the needle tends to slip into the anterior recess, risking an aortic perforation, or into the posterior recess, risking a free wall perforation. Sometimes, the needle may perforate the atrial septum and the free left atrial wall.

To minimize this risk, a clinician must be familiar with the anatomy of the atrial septum. There, however, is much anatomical variation in the intra-atrial septum. Consequently, a standard trans-septal needle may not always reach the fossa ovalis. For example, if a patient has a large right atrium, it is usually necessary to reshape the needle to give it a greater curvature. Thus, there is a need for novel and adaptable methods and devices for an accurate and safe trans-septal puncture.

### SUMMARY

The present teachings generally relate to devices, systems, and methods for treating heart failure. In one aspect, the present teachings provide a pressure relief shunt which can be retrieved, repositioned, adjusted, expanded, contracted, occluded, sealed, and/or otherwise altered. In some instances, a pressure relief shunt can be created at a proper location, for example, at or close to the fossa ovalis.

In another aspect, the present teachings provide a trans-septal puncturing device. In some embodiments, the trans-septal puncturing device is used to create an aperture, which by itself or in combination of a pressure relief shunt can be used to change the pressure in a heart chamber. In various embodiments, the trans-septal puncturing device includes one or more elements each independently selected from a puncturing element, a safety element, a tubular body, a locator, and a stabilizer, each of which is discussed in detail herein. In certain instances, a trans-septal puncturing device of the present teachings facilitates the location and puncturing of heart tissues at a desired location, for example, the fossa ovalis, while prevents or reduces inadvertent puncturing of heart tissues, including a free wall.

In another aspect, the present teachings provide methods of changing, for example, reducing, the pressure in a heart chamber. An example of the method includes locating a desired location on the atrial septum and puncturing the septum at the desired location. In some embodiments, the desired location is at or near the fossa ovalis. In some embodiments, the desired location is on the septum primum or the septum secundum.

Without intending to limit the scope of the present teachings, embodiments of the present teachings can have one or more desired properties. For example, some embodiments of the present teachings decrease the risk of inadvertent puncture of structures other than the atrial septum; some embodiments provide a better control of puncture within or near a desired location (e.g., the fossa ovalis), including preventing slippage of the puncturing element during the puncture and maintaining tracking through the sheath; some embodiments improve the ability of puncturing tough septal tissues or aneurysmal septa; some embodiments include elements that can be used to monitor or measure the pressure in a heart chamber or inject a dye into the heart chambers during or after an initial puncture; some embodiments reduce the risk of embolism (for example, by reducing skiving particulate, air, or thrombus); some embodiments provide visualization of the puncturing element, for example, by an echo technique; and other embodiments simplify trans-septal puncture procedures and/or reduce the necessity for user training. In addition, many embodiments of the present teachings require a minimal or justifiable cost increase.

### BRIEF DESCRIPTION OF THE DRAWINGS

FIG. 1 depicts the left atrium, the right atrium, and the fossa ovalis of a heart.

FIGS. 2A-2C are perspective views of an exemplary locating element of a trans-septal puncture device in accordance with the present teachings.

FIG. 3 is a perspective view of another exemplary locating element of a trans-septal puncture device in accordance with the present teachings.

FIG. 4 is a perspective view of another exemplary locating element of a trans-septal puncture device in accordance with the present teachings.

FIG. 5 is a perspective view of an exemplary stabilizing element of a trans-septal puncture device in accordance with the present teachings.

FIG. 6 is a perspective view of another exemplary stabilizing element of a trans-septal puncture device in accordance with the present teachings.

FIGS. 7A-7B are perspective views of an exemplary puncturing element with a safety element of a trans-septal puncture device in accordance with the present teachings.

FIG. 8 is a perspective view of another exemplary puncturing element with a safety element of a trans-septal puncture device in accordance with the present teachings.

FIG. 9 is perspective views of another exemplary trans-septal puncture system in accordance with the present teachings.

### DETAILED DESCRIPTION

Certain specific details are set forth in the following description and drawings to provide an understanding of various embodiments of the present teachings. Those with ordinary skill in the relevant art will understand that they can practice other embodiments of the present teachings without one or more of the details described below. While various processes are described with reference to steps and sequences in the following disclosure, the steps and sequences of steps should not be taken as required to practice all embodiments of the present teachings.

As used herein, the terms “radially outward” and “radially away” means any direction which is not parallel with the central axis. For example, with respect to a cylinder, a radial outward member could be a piece of wire or a loop of wire that is attached or otherwise operatively coupled to the cylinder and oriented at some angle greater than 0 relative to the central longitudinal axis of the cylinder.

As used herein, the term “lumen” means a canal, duct, generally tubular space or cavity in the body of a subject, including veins, arteries, blood vessels, capillaries, intestines, and the like. The term “lumen” can also refer to a tubular space in a catheter, a sheath, or the like in a device.

As used herein, the term “proximal” means close to the operator (less into the body) and “distal” means away from the operator (further into the body). In positioning a medical device inside a patient, “distal” refers to the direction away from a catheter insertion location and “proximal” refers to the direction nearer the insertion location.

The present teachings generally relate to devices used to create an aperture, or a hole, on the atrial septum. One aspect of the present teachings provides a device that can be used to locate an optimal trans-septal puncture site, for example, the fossa ovalis. Another aspect of the present teachings provides a trans-septal puncturing device that can stabilize the tissue to be punctured, limit area of puncturing, and/or prevent punctuating wire/needle slippage. Another aspect of the present teachings provides devices that can be used to pierce the septum from the right atrium to left atrium through an identified puncture site without inadvertently damaging tissue around the puncture site. In some embodiments, a trans-septal puncture is for subsequently implanting a device that can act as a left-to-right blood shunt and regulate blood flow between the left atrium and the right atrium. In various embodiments, a trans-septal puncturing is followed by removing septal tissue to form a left-to-right blood shunt.

Another aspect of the present teachings provides a trans-septal puncturing device having a puncturing element. In various embodiments, the puncturing element comprises a distal portion having a sharp tip. For example, the sharp point can be the tip of a needle or a wire. In some embodiments, the distal portion includes a solid needle. In some embodiments, the distal portion includes a hallow needle.

In various embodiments, at least the distal portion of the puncturing element is uniform and monolithic. In such embodiments, the distal portion is made of one material strong enough for puncturing tissues, yet flexible enough for percutaneous delivery inside the body. In some embodiments, the entire puncturing element is made of one material strong enough for puncturing tissues and flexible enough for percutaneous delivery inside the body. For example, the material can be stainless steel, titanium, aluminum, ceramic, alloy metal, carbon composite, and any composite thereof. In some embodiments, a part of or the entire puncturing element is made of an echogenic material, including a porous material, or is coated with an echogenic coating.

In various embodiments, where the distal portion of the puncturing element is made separately from the elongated body, a subsequent attachment is required. In some embodiments, such an attachment is achieved by a variety of means, including mechanical means, for example an interference connection or a threaded connection; energy means such as heat, laser, ultrasonic, or other types of welding; or by chemical means such as adhesive bonding. Other methods of attachment known to those ordinarily skilled in the art can also be incorporated.

In other embodiments, the puncturing element includes an elongated body. In some embodiments, the puncturing element includes a tubular body. In such embodiments, the tubular body is made of a flexible material. A person with ordinary skill in the art would understand that any materials, as long as they can achieve the desire property, can be used to make a puncturing element of the present teachings. For example, the material can be any polymeric material, including polyether-block co-polyamide polymers, for example Pebax™; polyethylene, polytetrafluoroethylene (EPTFE), fluorinatedethylenepropylene (FEP), polyurethane, mixture thereof, and the like, and any metallic material, including stainless steel, titanium, gold, platinum, copper, aluminum, or any alloy.

In various embodiments, the puncturing element includes a sharp distal tip. In some embodiments, the sharp distal tip has an outside diameter of equal or less than 0.020″. In some embodiments, the sharp distal tip has a tapered end. The sharp distal tip in such embodiments can have several advantages. Without intending to limit the scope of the present teachings, a sharp distal tip can be adapted to locate a desired puncture site or stabilize the puncturing element at a desired site. In addition, a sharp distal tip in conjunction with a section of a greater diameter can be used to dilate an initial small puncture and create an aperture with a desired size without creating any torn tissues, shiving particulates, or any other residues that can create an embolism.

In various embodiments, the distal portion of a puncturing element of the present teachings has an adjustable flexibility/stiffness. In some embodiments, the distal portion includes two or more segments with a first diameter and a second diameter. Without attempting to limit the scope of the present teachings to any particular theory, the first diameter provides a greater flexibility than the second diameter. In certain embodiments, at least two of the two and more segments are two sections of a continuous structure.

In various embodiments, by manipulating the proximal end, for example, the flexibility of the distal portion of a puncturing element is adjusted. In various embodiments, the manipulation of the proximal end is accomplished by a mechanical mechanism. In some embodiments, the mechanical mechanism is an actuator, a gear, a pulley, or the like.

Another aspect of the present teachings provide a safety element, for example, an incising stop element, that is capable of preventing a puncturing element as described herein from inadvertently puncturing heart tissues. In one aspect of the present teachings, a trans-septal puncturing device having a limited puncturing distance due in part to the safety element is provided. As a result, the risk of inadvertently puncturing tissues other than the atrial septum can be reduced or eliminated.

In various embodiments, the safety element is connected either directly or indirectly with the puncturing element. In some embodiments, the safety element and the puncturing member are made of a single material. For example, a nitinol or stainless steel tube can be wound into a safety element, for example, a spring, and one end of the tube can be ground into a sharp point as the puncturing element.

In some embodiments, there are one or more other elements between the safety element and a puncturing element as described herein. In certain embodiments, the safety element is proximal to the sharp tip of the distal portion of the puncturing element. In certain embodiments, the safety element is distal to the sharp tip of the distal portion of the puncturing element. In various embodiments, the safety element is independently operated from the puncturing element. For example, the safety element can be adjacent to the puncturing element.

Another aspect of the present teachings provides a locating element. In some embodiments, a locating element of the present teachings has a first configuration and a second configuration where at least one dimension of the second configuration is greater than that of the first configuration. Without attempting to limit the present teachings to any particular embodiment, the locating element can be used to identify an optimal puncture site. In some embodiments, the locating element includes a side arm, a plane, a ball, or the like at or close to the distal end of the elongated body.

In various embodiments, the locating element includes a side arm. For example, the locating element can include an elongated body, from which the side arm is attached, and the angle between the side arm and elongated body can change. In some embodiments, the locating element includes one or more side arms and an elongated body, from which the one or more side arms are attached. For example, the side arms and the elongated body can resemble an umbrella where the elongated body resembles the center shaft of the umbrella and the side arms resemble the ribs. When in use, in certain embodiments, the elongated body extends between the inferior vena cava and the superior vena cava, the one or more side arms extend from the elongated body and one of the side arms extends into and locates the fossa ovalis. In some embodiments, at least one of the side arms is retractable. For example, by extending a sheath over the elongated body and retracting the elongated body into the sheath, the side arms fold towards the elongated body and are retracted into the sheath.

In some embodiments, the locating element includes one or more side arms each independently having one end affixed to the elongated body (“fixed end”) and the other end extending radially outward (“free end”). In certain embodiments, at least one dimension of the fixed end is smaller than that of the free end. In certain embodiments, at least one dimension of the fixed end is greater than that of the free end. In certain embodiments, at least one dimension of the fixed end is the same as that of the free end. In certain embodiments, the side arm includes at least one dimension varying from one end to the other. In particular embodiments, the at least one dimension is greater at a section between the free end and the fixed end. For example, the at least one side arm can resemble a “bunny ear.”

In some embodiments, the locating element includes an elongated body having a distal end and a plane selected from a circular plane, an oval plane, a polygonal plane, and a part thereof, where the plane is attached at or close to the distal end of the elongated body. In some embodiments, the locating element includes a ball, for example, a spherical or ovoid ball. In certain embodiments, the locating element further includes an elongated body having a distal end where the ball as described herein is attached at or close to the distal end of the elongated body. In the above embodiments, the length of the one or more arms, the distance from the elongated body to a side of the plane, or the diameter of the ball determines the location of an optimal puncture site, i.e. the fossa ovalis. Accordingly, the location where a puncture is made can be adjusted by changing the length of the one or more arms, the size of the plane, or the diameter of the ball. In certain embodiments, a puncture at the limbus of the fossa ovalis is avoided.

Accordingly, an exemplary method of using such an embodiment includes advancing a locating element as described herein to a proximity of a desired location, for example, the fossa ovalis, so that the distal end of the locating element is placed near the center of the fossa ovalis.

In various embodiments, the locating element includes an elongated body having a longitudinal lumen. For example, a puncturing element as discussed herein can be advanced through the longitudinal lumen to a desired location on the atrial septum.

Another aspect of the present teachings provides a trans-septal puncturing device having a stabilizing element to limit the area exposed to the puncturing element. For example, the stabilizing element can be used to stabilize and/or center the trans-septal puncturing device in the fossa ovalis and prevent it from inadvertently injuring other parts of the heart. In various embodiments, the stabilizing element is also used as a locating element as described herein. In various embodiments, the stabilizing element includes an elongated body having a distal portion and one or more struts extending from the distal portion. In some embodiments, the one or more struts each includes a fixed end engaged to the elongated body and a free end configured to move radially away from the elongated body.

In various embodiments, the stabilizing element is slidably disposed within a longitudinal lumen of a sheath, wherein the struts are stowed radially along the elongated body of the stabilizing element. In other embodiments, the struts expand radially to form a supporting surface when the distal portion of the stabilizing element is exposed outside of the longitudinal lumen of a sheath. In some embodiments, the one or more struts form a supporting surface around the fossa ovalis or another desired location on the atrial septum.

In various embodiments, the stabilizing element includes a tubular member having a distal end. In some embodiments, the distal end stabilizes a trans-septal puncturing device described herein by temporarily engaging heart tissues at or near the fossa ovalis or another desired location on the atrial septum. In certain embodiments, the distal end of the stabilizing element includes an element in the shape of a funnel, which in certain instances can center and/or stabilize a puncturing element. For example, vacuum can be applied so as to temporarily engage the stabilizing element to heart tissues at a desired location site.

In certain embodiments, the distal end includes one or more struts, extending either inwards or outwards and optionally having a stabilization mechanism. The stabilization mechanism, for example, can include a barb, a hook, a needle, a suction cup, or the like. In certain embodiments, the stabilization mechanism includes one or more micro-electro-mechanical system (MEMS) needles. In particular embodiments, the stabilization mechanism includes a MEMS needle array. In various embodiments, by advancing the distal end of the stabilizing element against tissues at a desired site, the one or more struts pivot or deflect to engage heart tissues at the desired site. In such embodiments, after the struts are placed around the fossa ovalis, the stabilization mechanism assists the trans-septal puncturing device to maintain its place.

In some embodiments, the safety element, the locating element, the stabilizing element, or a combination thereof includes a first configuration when it is constrained in a sheath and a second configuration when it extends away from the sheath. The stabilizing element and the safety element at the second configuration, for example, can reside adjacent to or against the septum that separates the two atria. The locating element at the second configuration, for example, can reside inside the superior vena cava and/or in the right atrium.

In various embodiments, the safety element, the locating element, the stabilizing element, or a combination thereof in part or its entirety is made of an elastic material, a super-elastic material, or a shape-memory alloy which allows selected portions to distort into a generally straightened profile during a delivery process and resume and maintain its intended profile in vivo once it is deployed. For example, the safety element, the locating element, or the stabilizing element can manually be shaped to the desired deployment profile, heat set in an oven while constrained to such desired profile to memorize such a desired profile. In some embodiments, the safety element, the locating element, or the stabilizing element in part or entirety is made of stainless steel, nitinol, Titanium, Elgiloy, Vitalium, Mobilium, Ticonium, Platinore, Stellite, Tantalum, Platium, Hastelloy, CoCrNi alloys (e.g., trade name Phynox), MP35N, or CoCrMo alloys or other metallic alloys. Alternatively, in such other embodiments, part or all of the safety element, the locating element, or the stabilizing element is made of a polymer such as PTFE, UHMPE, HDPE, polypropylene, polysulfone, polymethane, Pebax®, or another biocompatible plastic.

In various embodiments, the safety element, the locating element, the stabilizing element, or a combination thereof includes a radio-opaque marker or is made in part or its entirety of a radio-opaque material. By using a visualization technique, including various echoing, x-ray, fluoroscopic, or magnetic resonance imaging techniques, a clinician can use the marker to show whether the trans-septal puncturing device reaches a proper location.

In various embodiments, the safety element, the locating element, and/or the stabilizing element of the present teachings includes a flexible body so that it can be delivered through the tortuous path inside the sheath. In some embodiments, the locating element further conforms the inside anatomy of the right atrium, so as to provide a clear indication/visualization of the location of the fossa ovalis.

Another aspect of the present teachings provides a trans-septal puncturing device having a monitoring element. In various embodiments, the monitoring element is a pressure wire or a sensor. For example, when the septum between the right and left atria is punctured, the pressure wire or sensor can be used to detect the pressure change in the right atrium and indicate the initial puncture. In various embodiments, the monitoring element is an injection port. Prior to, at, or after an initial puncture of the septum is made, a dye can be injected via the injection port and the breach of the dye into the left atrium can be used as an indication of the initial puncture.

Another aspect of the present teachings provides a trans-septal puncturing device. In various embodiments, the trans-septal puncturing device comprises a puncturing element as described herein slidably disposed in a longitudinal lumen of a sheath. In various embodiments, the trans-septal puncturing device comprises a safety element as described herein slidably disposed in a longitudinal lumen of a sheath. In various embodiments, the trans-septal puncturing device comprises a locating element as described herein slidably disposed in a longitudinal lumen of a sheath. In various embodiments, the trans-septal puncturing device comprises a stabilizing element as described herein slidably disposed in a longitudinal lumen of a sheath. In various embodiments, the trans-septal puncturing device comprises a monitoring element as described herein slidably disposed in a longitudinal lumen of a sheath.

One or more, if more are included in a trans-septal puncturing device as described herein, of the puncturing element, the safety element, the locating element, the stabilizing element, and the monitoring element can be disposed within a longitudinal lumen of a sheath. Thus, in some embodiments, the puncturing element, the safety element, the locating element, the stabilizing element, or the monitoring element, when more are included in the trans-septal puncturing device, each is slidably disposed within a different longitudinal lumen; in some other embodiments, two of the puncturing element, the safety element, the locating element, the stabilizing element, and the monitoring element independently are slidably disposed within one longitudinal lumen of a sheath; in other embodiments, three of the puncturing element, the safety element, the locating element, the stabilizing element, and the monitoring element independently are slidably disposed within one longitudinal lumen of a sheath; in other embodiments, four of the puncturing element, the safety element, the locating element, the stabilizing element, and the monitoring element independently are slidably disposed within one longitudinal lumen of a sheath; and yet in other embodiments, all five of the puncturing element, the safety element, the locating element, the stabilizing element, and the monitoring element independently are slidably disposed within one longitudinal lumen of a sheath.

The sheath as described herein can be a catheter, a part of a catheter, or one independent from a catheter. The terms “sheath” and “catheter” are thus used interchangeably. In various embodiments, the trans-septal puncturing device also includes a control mechanism, through which a clinician can manipulate the puncturing element, the safety element, the locating element, the stabilizing element, and/or the monitoring element.

In various embodiments, a trans-septal puncturing device comprises a puncturing element with a first needle. In some embodiments, the first needle is incapable of puncturing a tissue unless it is constrained within a lumen. In some embodiments, the first needle includes a lumen, within which a wire is slidably disposed. In some embodiments, when such a trans-septal puncturing device is withdrawn from the body, the wire is left to mark the aperture and guide subsequent device implantation or other procedures. In some embodiments, the first needle optionally includes a hollow tapered tip with a cutting section.

Another aspect of the present teachings provides a trans-septal puncturing device including an echogenic element. In various embodiments, the echogenic element is independent from each of the puncturing element, the safety element, the locating element, the stabilizing element, and the monitoring element. In various embodiments, the echogenic element includes an echogenic material used in making the puncturing element, the safety element, the locating element, the stabilizing element, or the monitoring element, each of which is described herein. In some embodiments, the echogenic material is an echogenic coating.

Another aspect of the present teachings provides a method of percutaneously puncturing tissues in the heart. In various embodiments, the method comprises providing a trans-septal puncturing device as described herein; advancing the trans-septal puncturing device into the right atrium; optionally locating the fossa ovalis or another desired site on the atrial septum using a locating element; optionally stabilizing the trans-septal puncturing device on the atrial septum at or around the fossa ovalis or another desired site on the atrial septum with a stabilizing element; piercing septal tissues at or around the fossa ovalis or another desired site on the atrial septum, and optionally preventing inadvertent damage to surrounding heart tissue with the safety element. In some embodiments, the method includes using a locating element to locate the fossa ovalis or another desired site on the atrial septum. In some embodiments, the method includes using a stabilizing element to stabilize the trans-septal puncturing device at or around the fossa ovalis or another desired site on the atrial septum. In some embodiments, the method includes using a puncturing element to puncture tissues on the atrial septum. In some embodiments, the method includes using a safety element to prevent the puncturing element from damaging tissue around the fossa ovalis or another desired site on the atrial septum.

Depending upon the configuration of the trans-septal puncturing device used in the method, each of the puncturing element, the safety element, the locating element, the stabilizing element, and the monitoring element, when one or more are included in the trans-septal puncturing device, can be advanced into the heart simultaneously or sequentially and retracted simultaneously or sequentially. Accordingly, in some embodiments, the method includes advancing a puncturing element to the heart. In some embodiments, the method includes advancing a locating element into the heart. In some embodiments, the method includes advancing a safety element into the heart. In some embodiments, the method includes advancing a monitoring element to the heart. In other embodiments, the method includes advancing a stabilizing element into the heart.

Similarly, in some embodiments, the method includes retracting a puncturing element from the heart. In some embodiments, the method includes retracting a locating element from the heart. In some embodiments, the method includes retracting a safety element from the heart. In some embodiments, the method includes retracting a monitoring element from the heart. In other embodiments, the method includes retracting a stabilizing element from the heart.

In particular embodiments, a method of the present teachings includes providing a delivery sheath and a trans-septal puncturing device, wherein the delivery sheath comprises a distal end and a longitudinal lumen, the trans-septal puncturing device is slidably disposed within the longitudinal lumen of the delivery sheath, and the trans-septal puncturing device comprises a puncturing element with a sharp tip at a distal end, a locating element, and a stabilizing element slidably disposed within an elongated lumen of the delivery sheath; advancing the sheath holding the trans-septal puncturing device to a proximity of the fossa ovalis or a desired location on the atrial septum; retracting the sheath proximally; deploying the locating element at or near the fossa ovalis or a desired location on the atrial septum, deploying the stabilizing element against the fossa ovalis or a desired location on the atrial septum; advancing the puncturing element distally so that the sharp tip at the distal end of the puncturing element pierces the fossa ovalis (10) or the desired location on the atrial septum; deploying the safety element to stop or limit the movement of the puncturing element; retracting the locating element; retracting the stabilizing element; retracting the puncturing element proximally; allowing the distal portion of the trans-septal puncturing device to slide back into the delivery sheath.

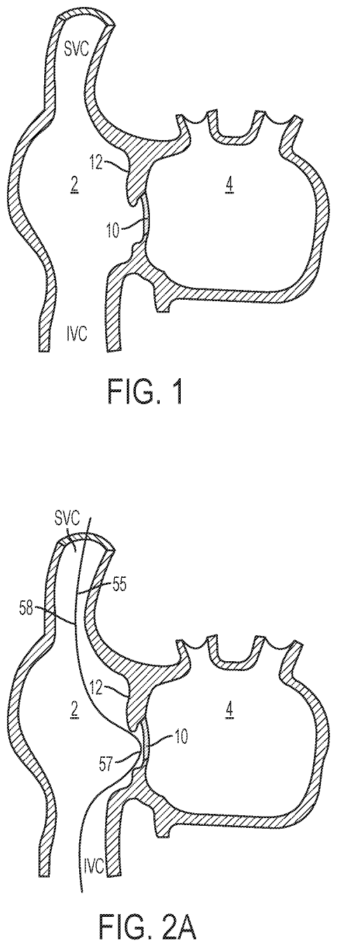

The following description refers to FIGS. 1 to 9. A person with ordinary skill in the art would understand that the figures and description thereto refer to various embodiments of the present teachings and, unless indicated otherwise by their contexts, do not limit the scope of the attached claims.

FIG. 1 is a diagram of a heart and it depicts the right atrium (2) and the left atrium (4). After a child is born, the septum primum and septum secundum fuse together, resulting in the atrial septum. On the atrial septum there is a depression, anatomically called the fossa ovalis (10). An incomplete fusion of the septum primum and septum secundum results in a patent foramen ovale (PFO). In this instance, without attempting to limit the scope of the present teachings, it can be advantageous to control the trans-septal puncture on the septum primum or the septum secundum.

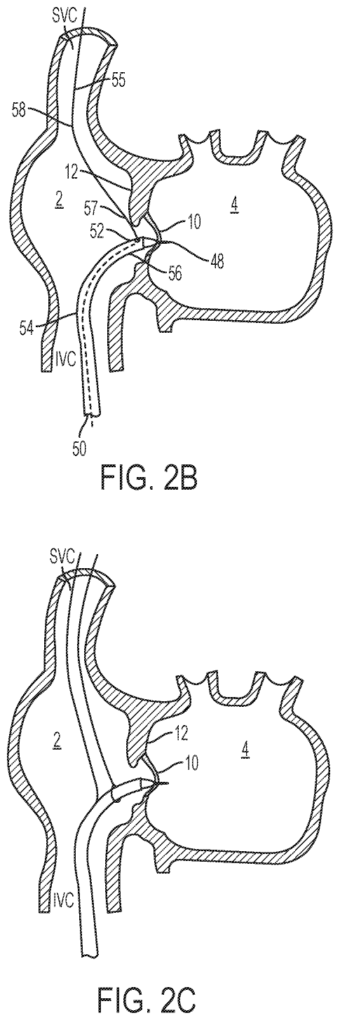

According to one embodiment of present teaching, a clinician uses a locating mechanism to locate the fossa ovalis (10) before piercing the septum tissue with a needle. FIGS. 2-4 illustrate various embodiments of locating elements. In some embodiments, a locating element is operably joined to the puncturing wire/needle. In some embodiments, a locating element also functions to identify the optimum puncture location and stabilize the puncturing wire/needle during the tissue piercing.

FIGS. 2A-2B depict an exemplary trans-septal puncturing system for locating and piercing the fossa ovalis (10) according to the present teachings. As shown in FIG. 2B, the device (44) includes an elongated body (54) having a proximal portion (not shown), a distal portion (56), and an axial lumen (50) extending in between. The elongated body (54) further includes a small opening (52) on the luminal surface at the distal portion (56) of the elongated body (54) near its distal end. In some embodiments, the elongated body (54) has a sharp tissue piercing distal tip (48) adapted to pierce the atrial septum (12). In other embodiments, the elongated body (54) is a delivery conduit for delivering a tissue puncturing wire/needle to the puncture site. FIG. 2B further shows that a locating wire (58) extends distally inside the lumen (50) of the elongated body (54) from its proximal end, exits the side opening (52). One skilled in the art should understand the locating wire (58) could join the elongated body via a monorail manner. According to some embodiments, the locating wire (58) has a pre-formed curve at a portion (57) proximal to its distal portion (55), where such a portion is adapted to remain inside the right atrium.

According to some embodiments, the trans-septal puncturing device is percutaneously delivered into the right atrium via a delivery sheath. Referring back to FIG. 2A, the trans-septal puncturing device has a first locating profile, where the distal portion (55) of the locating wire (58) reaches inside the superior vena cava (40), the curved portion (57) of the locating wire (58) resides inside the right atrium, and the pre-formed curve bends toward the atrial septum (12). Referring to FIG. 2B, the trans-septal puncturing device has a second profile, where the distal portion (55) of the locating wire (58) reaches inside the superior vena cava (40), the distal portion (56) of the trans-septal puncturing device extends distally, causing the portion of the locating wire (58) inside the right atrium bent toward the atrial septum (12), and the tissue piercing tip (48) is placed at or near the atrial septum (12).

In some embodiments, the locating wire (58) is configured to locate the fossa ovalis (10). Upon advancing the locating wire inside the right atrium, a clinician pushes the locating wire (58) distally so that the distal portion (55) of the locating wire (58) further extends distally and reaches inside the superior vena cava (40) and the curved portion (57) of the locating wire (58) extends inside the right atrium. At this point, the clinician can manipulate the locating wire (58) so that it (58) is curved toward the atrial septum (12). The result is that the locating wire (58) bends toward the atrial septum (12) and marks the general anatomy of the atrial septum (12). The fossa ovalis (10) can be located at the greatest curvature on the locating wire (58). Maintaining the distal portion (55) of the locating wire (58) inside the superior vena cava (40), the clinician advances the elongated body (54) to track the locating wire (58) until it reaches the portion of the greatest curvature and advances the puncturing element to pierce the septum at the fossa ovalis (10).

Although the locating element is shown as a wire (58) in FIGS. 2A-2B, one ordinarily skilled in the art would understand that the locating element can be a tubular member with a side opening, through which a puncturing wire/needle optionally with a delivery sheath extends toward the fossa ovalis as illustrated in FIG. 2C. The curve portion of the locating element in FIGS. 2A-2C can be pre-formed as discussed herein. The curve portion of the locating element can also be mechanically actuated after the locating element is delivered inside the right atrium.

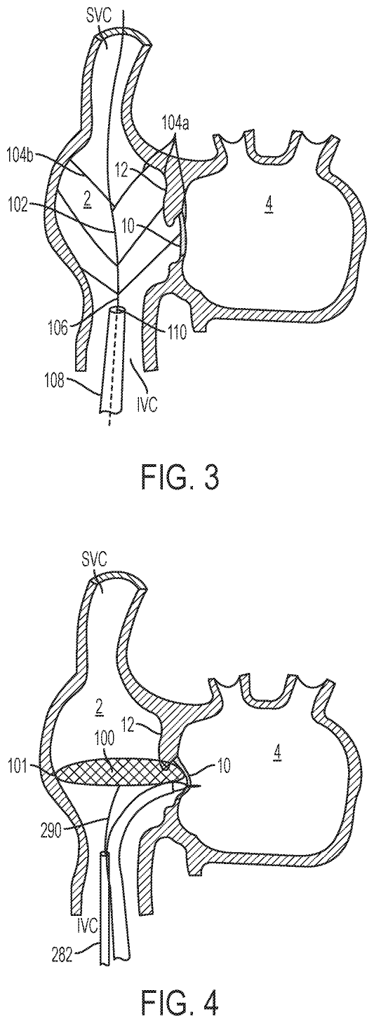

Another exemplary trans-septal puncturing system for locating the fossa ovalis (10) is depicted in FIG. 3. The trans-septal puncturing system for locating the fossa ovalis (10) includes a right atrium marker system. The system includes a right atrium marker (102), a septal puncture wire/needle (not shown), and a sheath (108) having a proximal end, a distal end, and a central lumen (110). The right atrium marker (102) includes an elongated body (106) with a proximal portion (not shown) and a distal portion. The distal portion of the elongated body (106) has a plurality of side arms (104a and 104b) pivoting radially outward as shown in FIG. 6. Each of the side arms (104a and 104b) has a free end and a fixed end and is affixed to the elongated body by the fixed end. The right atrium marker system has a first delivery profile and a second deployment profile. In the first delivery profile, the right atrium marker (102) is slidably disposed in the sheath (108) and the side arms (104a and 104b) are pivot distally and radially inward towards the elongated body (106). In the second deployment profile, the distal portion of the right atrium marker (102) extends distally outside of the sheath (108) and the side arms (104a and 104b) expand radially outward with their free ends touching the surrounding heart wall.

According to one embodiment, the location of the free end of each of the side arms is monitored or visualized, for example, by an echoing technique, an X-ray technique, a fluoroscopy, a magnetic resonance imaging technique, or the like. A clinician can then determine the location of the fossa ovalis (10) by examining the radial distance of each free end. In some embodiments, upon determining the location of the fossa ovalis (10), a clinician advances a puncture wire/needle to such a location to puncture the tissue. When needed, the right atrium marker (102) can be retracted by extending the sheath (108) distally over the distal portion of the right atrium marker (102), causing the sides arms to collapse distally radially inward and sliding into the sheath (108). Alternatively, the right atrium marker (102) can be withdrawn proximally, also causing the side arms to collapse distally radially inward and sliding into the sheath (108). The resulting trans-septal puncturing device can be removed from the body or repositioned inside the right atrium if necessary.

In some embodiments, the locator includes 2-8 groups of side arms. In another embodiment, each group of the side arms includes 2-8 side arms. As the right atrium marker system is deployed inside the right atrium, the side arms extend radially outward to touch the boundary of the right atrium. One skilled in the art would understand that more side arms in these instances can give a move accurate right atrium marking.

FIG. 4 depicts another exemplary trans-septal puncturing system for locating the fossa ovalis (10). This embodiment includes a sheath (282) and a locator (100). The locator has an elongated body (290) with a radially expanded distal end (101). The elongated body (290) can be manipulated at its proximal end by a clinician. The circumference edge of the radially expanded distal end (101) of the locator (100) deflects when it touches the surrounding heart wall. Similar to the right atrium marker system described above, the locator (100) has an elongated profile when it is constrained inside a delivery sheath (282) and a radially expanded profile when it is deployed. FIG. 4 illustrates a locator deployed inside the right atrium. In one embodiment, the locator resembles an umbrella with spokes. In another embodiment, the locator resembles a braided wire mesh having a substantially flat surface. To locate the fossa ovalis (10), a clinician can extend or retract the locator (100) and observe its edge deflection via a visualization technique. The location with the least deflection on the atrial septum (12) is where the fossa ovalis (10) is located. Upon locating fossa ovalis (10), a clinician can then advance a separate puncture wire/needle to such a location and puncture the tissue.

According to one embodiment, the locating wire, the pivotable side arms of the right atrium marker, and the expanded distal end of the locator discussed herein can be made from an elastic material, a super-elastic material, or a shape-memory alloy. And thus, the transition from an elongated delivery profile to an expanded deployed profile is accomplished by elastic recovery or thermal-shape transformation.

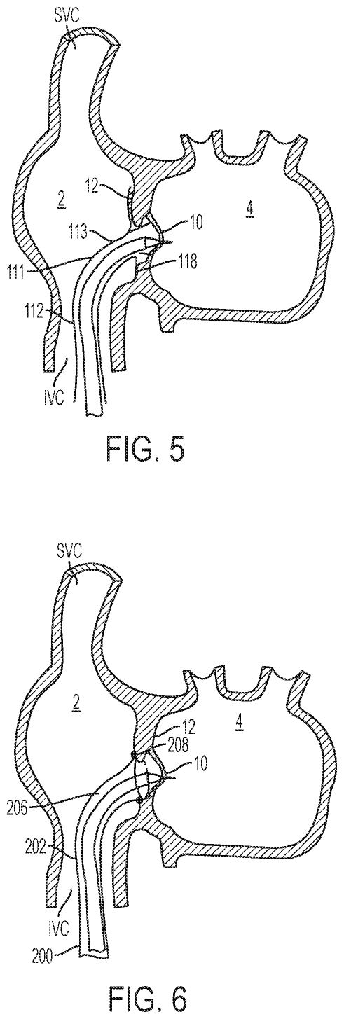

FIGS. 5-6 illustrate various embodiments of stabilizing elements according to the present teachings. A stabilizing element is used to stabilize the puncturing wire/needle during a tissue puncture. In some embodiments, the stabilizing element is configured to engage the septal tissue at or near a desirable puncturing location and prevent the slippage of the puncture wire/needle. In these embodiments, the puncturing wire/needle is disposed within the central lumen of the stabilizing element so that it is confined within a limited space.

FIG. 5 depicts an exemplary stabilizer (112) according to the present teachings. According to FIG. 5, the stabilizer (112) has an elongated body with a proximal end extending outside of the body, a distal end (113), and a central lumen (111). A clinician can manipulate such stabilizer from the proximal end. The stabilizer (112) includes a plurality of struts (118) at its distal end (113). In a delivery configuration, the struts are folded radially inward distally or proximally and are stowed within a sheath (not shown). In a deployed configuration, the struts expand radially outward, resulting in a tissue contacting surface.

In some embodiments, there are at least 2-12 struts distributed at the distal end of the stabilizer (112). In certain embodiments, the at least 2-12 struts are evenly distributed. According to some embodiments, the struts are flexible and are capable of deflecting when contacting the heart tissue. According to other embodiments, the tissue contacting surface of at least one of the struts (118) can include a securing element. In certain embodiments, the securing element includes an array of Velcro-type elements, an array of the MEMS (Micro-Electro-Mechanical Systems) needles, or a plurality of barbs. Such a securing element can be used to securely engage the tissue when the struts are pressed on the atrial septum (12).

According to some embodiments, a puncturing wire/needle is slidably disposed within the lumen (111) of a stabilizer (112). Once the stabilizer is delivered inside the right atrium, a clinician can either withdraw the sheath proximally or advance the stabilizer (112) distally so that the struts (118) extend outside of the sheath (not shown) and expand radially outward. A clinician can then manipulate the stabilizer (112) to allow the struts (118) to engage the atrial septum at or near the fossa ovalis (10). A puncturing wire/needle can then be advanced distally inside the lumen of the stabilizer (112) to puncture the tissue near the radial center of the struts (118). To remove a stabilizer (112), a clinician pulls the stabilizer (112) so that the struts descend from the tissue and retracts the stabilizer proximally so that the struts collapse inward radially and back inside the sheath (114). The stabilizer (112) can then be removed or repositioned if necessary.

FIG. 6 illustrates another exemplary stabilizing element. Similar to what's described above, this particular stabilizer (200) also includes an elongated body (202) with a proximal end (not shown), a distal end (208), and a central lumen (206). A clinician can manipulate the stabilizer via the proximal end. The stabilizer (200) further includes a tissue engaging distal end (208). The tissue engaging distal end (208) engages the septal tissue to form a seal. In some embodiments, the stabilizer (200) is advanced to a proximity of the atrial septum (12) in the right atrium and the distal end (208) is placed at or near a desired puncture site. Vacuum is applied and the distal end (208) of the stabilizer engages the septal tissue. A puncturing wire/needle is advanced inside the lumen (206) of the stabilizer (200) and punctures the tissue at or near the radial center of the stabilizer (200). To remove the stabilizer (200), a clinician stops the vacuum and retracts the stabilizer (200) proximally either back into a sheath (not shown) or directly outside of the body. In some embodiments, the distal end (208) of the stabilizer (200) resembles the shape of a ring, a cup, a segment ring, a cone, a funnel, or another shape with its size profile suitable for creating a seal to the tissue.

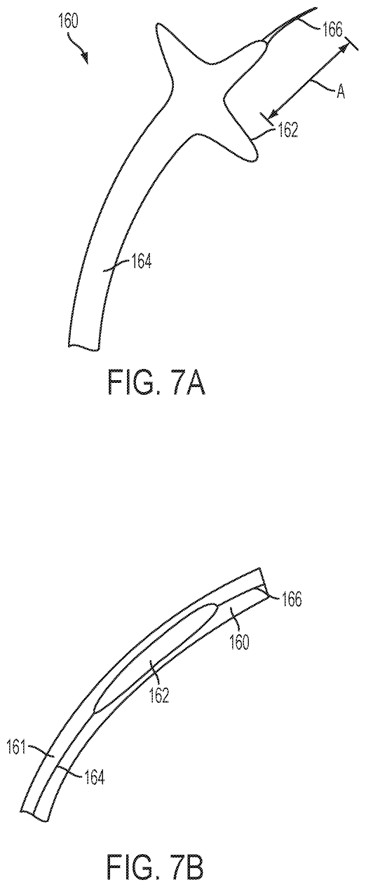

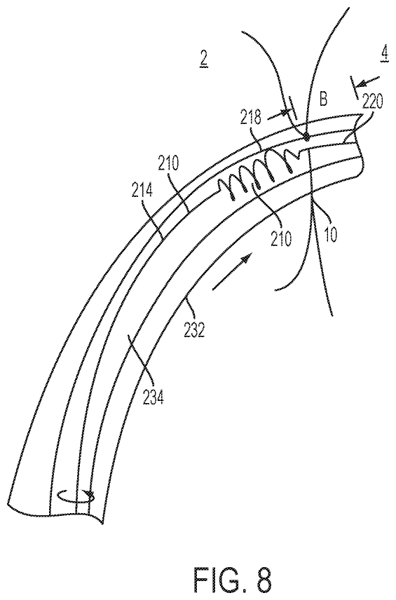

FIGS. 7-8 illustrate various embodiments of the puncturing elements associated with safety elements according to the present teachings. These puncturing elements are capable of piercing the septal tissue while avoiding inadvertently damaging nearby tissues. In some embodiments, the puncturing element has an elongated body with a sharp distal tip. The sharp distal tip can have the same diameter as the elongated body. The puncturing element can also taper toward its sharp distal tip. In some embodiments, the safety element associated with the puncturing element limits the distal movement of the sharp tip.

FIGS. 7A-B depict several exemplary puncturing elements of the present teachings. In one example, a puncturing element (160) comprises an elongated body (164) with a sharp distal tip (166) and one or more incision stopping elements (162). The one or more incision stopping elements are proximal to the distal tip (166) of the puncturing element (160). In some embodiments, there is a minimum distance “A” between the incision stopping elements (162) and the sharp distal tip (166), as shown in FIG. 7A. The puncturing element (160) has a delivery profile inside a sheath (161) as illustrated in FIG. 7B and a deployed profile as illustrated in FIG. 7A. In some embodiments, the incision stopping elements (162) of the puncturing element (160) are formed by cutting slits on a tube and compressing the tube. Alternatively, the incision stopping elements (162) of the puncturing element (160) are formed by radially expandable wires structures, flaps, strips, or other shape and forms suitable for producing a large surface. One ordinarily skilled in the art would understand that FIGS. 7A-7B show embodiments of the present teachings and should not be construed as limiting to the scope of the invention.

As illustrated in FIG. 7B, in a delivery profile, the incision stopping elements (162) of the puncturing element (160) has an elongated and narrow profile, suitable to be disposed inside a sheath (161). This particular profile can be created either by folding the incision stopping elements (162) radially inward or by stretching longitudinally. In the deployed profile, as shown in FIG. 7A, the incision stopping elements (162) of the puncturing element (160) expand radially outward to forming an enlarged portion. This portion has a cross section significantly greater than the distal tip. The transitioning between the delivery profile and the deployed profile is accomplished either by elastic recovery or thermal shape transformation.

In one embodiment, the distal tip (166) of the puncturing element (160) is configured to pierce through the septal tissue. In use, the distal portion puncturing element (160) is delivered inside the right atrium by a sheath (161) and the incision stopping elements (162) is pushed outside of the distal end of the sheath. The incision stopping elements expand radially to assume its expanded deployed profile. A clinician further extends the puncturing element (160) distally, allowing the sharp distal tip (166) of the puncturing element (160) to pierce the atrial septum. The extension of the puncturing element (160) is stopped when a clinician feels or visualizes that the incision stopping elements (162) is pushed against the atrial septum (12). Thus, the incision stopping element prevents the puncturing member from inadvertently advancing further and damaging the left atrial wall. In some embodiments, the length “A” is in the range of 1 mm-20 mm.

In some embodiments, to remove a puncturing element (160), a clinician retracts it (160) proximally back to the sheath (161), the expanded incision stopping element is forced back to its narrowed profile, and the sharp distal tip is withdrawn inside the sheath. The whole system is then removed from the body.

FIG. 8 depicts another exemplary puncturing element (210) with a safety element (218) according to the present teachings. This particular atraumatic puncturing element (210) includes an elongated body (214) with a sharp distal tip (220) and a helical portion (218) proximal to the distal tip (240) of the elongated body (214). Accordingly, the helical portion (218) can have a continuously increasing (e.g., in a linear or polynomial fashion) outside diameter or several staged outside diameters throughout its length. The proximal end of the exemplary atraumatic puncturing element (210) can be connected with a control mechanism (not shown), from which a clinician can control the puncturing. In some embodiments, there is a minimum distance “B” between the distal end of the helical portion (218) and the sharp distal tip (220), as shown in FIG. 8. In some embodiments, the length “B” is in the range of 0 mm-20 mm.

In some embodiments, the helical portion (218) is to stop further distal advancement of the distal tip (220), thereby preventing the distal tip (220) from damaging unintended area of the heart. In another embodiment, the helical portion (218) functions like an auger, which is to incising the septal tissue when a clinician torques it at its proximal end as illustrated in the arrow on FIG. 8. In yet another embodiment, the helical portion (218) is configured to be very flexible so that when it is outside of the sheath, it bends radially and directs the distal sharp tip (220) away from further damaging the left atrial wall.

Similar to what has been described in accordance with FIG. 7, the puncturing element (210) is delivered via a sheath (232) by sliding through its longitudinal lumen (234). Once inside the right atrium, the distal portion of the puncturing element (210) is advanced toward the atrial septum (12), so that the distal tip (220) of the puncturing element (210) engages the atrial septum (12). As the puncturing element (210) advances until the distal end of the helical portion (218) contacts the tissue, the helical portion (218) reduces or prevents the puncturing element (210) from lunging through the aperture and puncturing nearby heart tissues.

In some embodiments, a clinician can use a control mechanism (222) to advance the helical portion (218) of the puncturing element (210) through the septal tissue by torqueing the puncturing element (210). Upon entering the left atrium, in some embodiments, the flexibility of the helical portion (218) makes it deflect away from the long axis of the puncturing element (210) and away from the left atrium free wall.

In some embodiments, to remove the puncturing element (210), a clinician retracts the puncturing element (210) proximally so that the helical portion (218) of the puncturing element (210) stretches longitudinally and retracts to the lumen (234) of the sheath (232). In another embodiment, a clinician torques the puncturing element (210) backward so that the helical portion (218) of the puncturing element (210) retracts proximally through the atrial septum (12). When the helical portion (218) is entirely inside the right atrium, a clinician then retracts the puncturing element (210) proximally so that it (210) retracts into the lumen (234) of the sheath (232). With the sharp distal tip (220) is held inside the sheath (232), the whole system is then removed from the body.

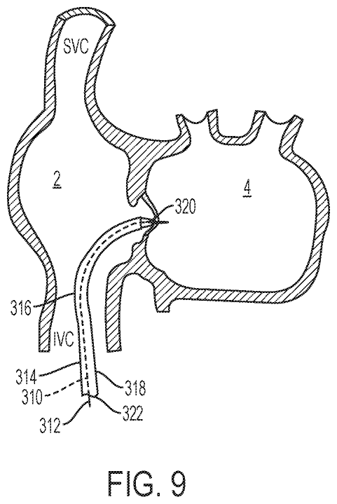

FIG. 9 illustrates an embodiment of the atraumatic puncturing system (310) according to the present teachings. This particular atraumatic puncturing system (310) includes a sheath (314) with a distal tip (320) and piercing wire (312). In one embodiment, the sheath (314) has a proximal portion (not shown) remaining outside of the body, a distal portion (316), an elongated body (318) between the proximal portion and the distal portion (318), and a central lumen (322) within the elongated body. In some embodiments, the distal end of the sheath (314) can be turned, rotated, or deflected. The deflectability or steerability of the sheath (314) allows a clinician to manipulate the distal end of the sheath (314) from outside of the body and advance it to a puncture location identified via visualization technique. Design and construction of a steerable and deflectable sheath are well known to those with ordinary skill in the art. One skilled in the art should understand that although a deflectable and/or steerable sheath (314) is described here, other types of the sheath with flexible, bendable deflectable distal end could also be incorporated to achieve the purpose of present teaching. Thus, the disclosure here should not be viewed as limiting.

In one embodiment, a piercing wire (312) slidably disposed within the lumen (322) of the sheath (314). In some embodiments, the piercing wire (312) tracks through lumen (322) of the sheath (314), with the distal end of the piercing wire (312) held inside the lumen (322) of the sheath while the whole system advanced toward the puncture site. In another embodiment, the piercing wire (312) is advanced from the proximal end of sheath (314) toward the distal tip (320) of the sheath (314) after the sheath (314) is positioned in place.

In some embodiments, via a visualization technique described herein, a clinician can manipulate the distal portion of the sheath (314) to bend toward the atrial septum. In some embodiments, a clinician can manipulate the sheath (314) so that its distal end can make contact with the atrial septum. A clinician can identify the puncture location, for example, the fossa ovalis, either by a visualization technique, or by other feedback methods known to those skilled in the art.

In one embodiment, the puncturing system (310) has a delivery profile, where the distal end of the piercing wire (312) is held inside the sheath (314). In some embodiments, the puncturing system (310) has a deployed profile, where the distal end of the piercing wire (312) extends distally outside of the distal tip (320) of the sheath (314). After the puncturing system (310) is delivered inside the right atrium and the distal tip (320) makes contact and identifies the puncture location, the piercing wire (312) is then pushed distally to cross the septal tissue.

In some embodiments, the locating element, stabilizing element, and safety element as disclosed above could be used in combination with the puncturing system disclosed in accordance with FIG. 9.

To remove the puncturing system (310) from crossing the atrial septum (12), a clinician simply retracts the piercing wire (312) so that the distal end of the piercing wire (312) returns inside the sheath (314). The puncturing system (310) retracts proximally and the whole system is then removed from the body.

One skilled in the art should understand that the present teachings are capable of being practiced individually or in combination. Furthermore, it is to be understood that the phraseology and terminology employed herein is for the purpose of description and should not be regarded as limiting.

Unless otherwise defined, all technical and scientific terms used herein have the same meaning as commonly understood by one of ordinary skill in the art to which these present teachings belong. Methods and materials similar or equivalent to those described herein can be used in the practice or testing of the present teachings. In case of conflict, the patent specification, including definitions, will control. In addition, the materials, methods, and examples are illustrative only and not intended to be limiting.

We claim:

1. A trans-septal puncturing system configured to mark an internal contour of a right atrium before puncturing a fossa ovalis, the system comprising
a right atrium marker having an elongated body with a plurality of side arms, wherein the right atrium marker has an collapsed profile with all the side arms of the right atrium marker radially contracted inward next to the elongated body, and an expanded profile with all the side arms of the right atrium marker radially expanded outward away from the elongated body, and wherein a free end of each of the side arms touches a heart wall inside the right atrium when the right atrium marker expands inside the right atrium,
a monitoring element configured to visualize a radial distance of each of the side arms, and
a puncturing element configured to pierce the fossa ovalis.

2. The trans-septal puncturing system of claim 1, wherein the right atrium marker further includes a sheath for the collapsed right atrium marker slidably disposed within.

3. The trans-septal puncturing system of claim 1, wherein the puncturing element comprises an elongated body with a sharp distal tip and an incision stopping element proximal to the distal tip.

4. The trans-septal puncturing system of claim 3, wherein the puncturing element further comprises a sheath, wherein the puncturing element has an elongated profile with the incision stopping element radially collapsed when inside the sheath, and an expanded profile with the incision stopping element radially expanded when outside of the sheath.

5. The trans-septal puncturing system of claim 4, wherein the radially expanded incision stopping element has a cross section significantly greater than the distal tip of the puncturing element.

6. The trans-septal puncturing system of claim 4, wherein as the distal tip of the puncturing element advances across a atrial septum from a right atrium to a left atrium to a first distance, and the incision stopping element extends outside of the sheath and resumes its radially expanded profile inside the right atrium.

7. The trans-septal puncturing system of claim 6, wherein as the incision stopping element pushes against the atrial septum, a distal movement of the distal tip of the puncturing element is stopped.

8. The trans-septal puncturing system of claim 1, wherein the puncturing element comprises an elongated body with a sharp distal tip and a helical portion proximal to the distal tip.

9. The trans-septal puncturing system of claim 1, wherein the helical portion of the puncturing element is configured to reduce a distal force to the distal tip of the puncturing element, and thereby preventing the distal tip of from puncturing nearby heart tissue.

10. The trans-septal puncturing system of claim 1, wherein the trans-septal puncturing system further comprises a stabilizing element configured to engage an atrial septum tissue adjacent the fossa ovalis, wherein the stabilizing element comprises an elongated body with a central lumen and a plurality of struts at a distal end of the elongated body, wherein the struts of the stabilizing element folds radially inward forming an elongated delivery configuration, and the struts of the stabilizing element expand radially outward forming an expanded tissue contacting profile.

11. A trans-septal puncturing system configured to puncture a fossa ovalis, the system comprising
a stabilizing element configured to engage an atrial septum adjacent the fossa ovalis, wherein the stabilizing element comprises an elongated body with a central lumen and a plurality of struts at a distal end of the elongated body, wherein the struts of the stabilizing element folds radially inward forming an elongated delivery configuration, and the struts of the stabilizing element expand radially outward forming an expanded tissue contacting profile;
a puncturing element having an elongated body with a sharp distal tip, and
wherein the puncturing element is slidably disposed within the central lumen of the stabilizing element.

12. The trans-septal puncturing system of claim 11, wherein the stabilizing element has at least two struts evenly distributed at the distal end of the elongated body.

13. The trans-septal puncturing system of claim 11, wherein as the struts of the stabilizing element engage the atrial septum tissue adjacent the fossa ovalis, a distal movement of the distal tip of the puncturing element is limited toward the direction of the fossa ovalis.

14. The trans-septal puncturing system of claim 11, wherein the puncturing element further comprises an incision stopping element proximal to the distal tip, wherein the incision stopping element has a radially collapsed profile when inside the central lumen of the stabilizing element, and a radially expanded profile when outside of the central lumen of the stabilizing element, and wherein the radially expanded stabilizing element has a greater cross section profile than the distal tip of the puncturing element.

15. The trans-septal puncturing system of claim 14, wherein as the distal tip of the puncturing element advances across a atrial septum from a right atrium to a left atrium to a first distance, and the incision stopping element extends outside of the stabilizing element and resumes its radially expanded profile inside the right atrium.

16. The trans-septal puncturing system of claim 15, wherein as the incision stopping element pushes against the atrial septum and a distal movement of the distal tip of the puncturing element is stopped.

17. The trans-septal puncturing system of claim 11, wherein the puncturing element further comprises a helical portion proximal to the distal tip.

18. The trans-septal puncturing system of claim 17, wherein the helical portion of the puncturing element is configured to reduce a distal force to the distal tip of the puncturing element, and thereby preventing the distal tip of from puncturing nearby heart tissue.
